(19) United States
(12) Patent Application Publication
(10) Pub. No.: US 2017/0354478 A1
(43) Pub. Date: Dec. 14, 2017

(54) MEDICAL DEVICE IDENTIFICATION SYSTEM

(71) Applicant: BIOPRO, INC., Port Huron, MI (US)

(72) Inventors: Joseph ADAMS, Howell, MI (US); Paul ZWIRKOSKI, Pinckney, MI (US); David MRAK, North Street, MI (US)

(21) Appl. No.: 15/617,360

(22) Filed: Jun. 8, 2017

Related U.S. Application Data
(60) Provisional application No. 62/347,149, filed on Jun. 8, 2016.

(51) Int. Cl. A61B 90/90; A61B 17/86; G09F 3/02

(52) U.S. Cl. CPC A61B 90/90; G09F 3/02; A61B 17/86

(57) ABSTRACT

A medical device identification system includes an attachment feature including a plurality of spaced-apart elongate members configured for removable attachment to a medical device. The system also includes an identification feature connected to the attachment feature and having a surface for receiving identifying indicia related to the medical device.

### CROSS-REFERENCE TO RELATED APPLICATIONS

This application claims the benefit of U.S. provisional application Ser. No. 62/347,149 filed Jun. 8, 2016, which is hereby incorporated herein by reference in its entirety.

### TECHNICAL FIELD

The present disclosure relates to a medical device identification system.

### BACKGROUND

Because of the need to trace materials and manufacturing processes, and generally have a complete history of the life of a medical device, various ways of providing identifying indicia for the medical device have been used. These may include, for example, etching or engraving a device with a lot number, a serial number, or some other identifying label. Although this system of identification and labeling has been successfully used for larger medical devices, very small medical devices have typically been identified by a lot or batch number only, and each individual device did not carry with it its own unique identifier. Examples of these small medical devices include tissue fasteners, such as bone screws, and small bone plates used to repair bone fractures. These small devices are typically not large enough to engrave or etch, or to otherwise label in such a way that the identifying information will remain with them until they are implanted. Therefore, a need exists for a system for medical device identification that can be effectively used with small devices.

### SUMMARY

At least some embodiments described herein may include a medical device identification system that includes an attachment feature including a plurality of spaced-apart elongate members configured for removable attachment to a medical device. The system may also include an identification feature connected to the attachment feature and including a surface for receiving identifying indicia related to the medical device.

In at least some embodiments, the attachment feature may include two or more of the elongate members, where each of the elongate members forms a portion of a cylindrical or other-shaped housing configured to receive a tissue fastener. The attachment feature may further include a release feature configured to cooperate with a tool for attaching the tissue fastener to tissue, such that inserting the tool into the tissue fastener when the tissue fastener is disposed in the housing actuates the release feature to release the tissue fastener from the housing. In at least some embodiments, the attachment feature may include two of the elongate members, each of the elongate members including a clip transversely extending outward therefrom.

### BRIEF DESCRIPTION OF THE DRAWINGS

FIG. 1 shows a perspective view of a medical device identification system in accordance with embodiments described herein;

FIG. 2 shows a cross-sectional view of the medical device identification system shown in FIG. 1;

FIG. 3 shows the medical device identification system of FIG. 1 including a bone screw and a driver attached to the screw;

FIG. 4 shows a perspective view of a medical device identification system in accordance with embodiments described herein;

FIG. 5 shows the medical device identification system of FIG. 4 attached to a bone plate; and

FIG. 6 shows a cross-sectional view of the medical device identification system and bone plate illustrated in FIG. 5.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

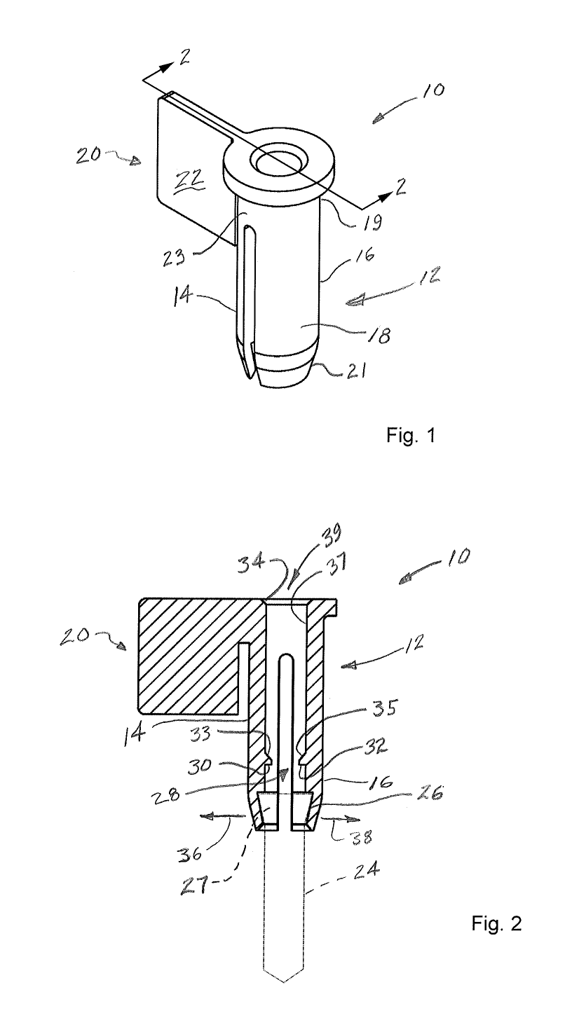

FIG. 1 shows a medical device identification system 10 in accordance with at least some embodiments described herein. The identification system 10 is configured to allow identifying indicia to be associated with a small medical device, for example, to meet medical device tracking requirements, or to otherwise identify the medical device. As used herein, “medical device” may refer to an implantable device; however, it may also refer to external devices or associated instrumentation. The identification system 10 includes an attachment feature 12, which, in this embodiment, includes a pair of spaced-apart elongate members 14, 16. The elongate members 14, 16 are configured for removable attachment to a medical device, and in particular a tissue fastener such as a bone screw. In the embodiment shown in FIG. 1, the elongate members 14, 16 form a portion of a cylindrical housing 18 configured to receive a bone screw. In other embodiments, more than two elongate members may form a portion or all of a housing, which may or may not be cylindrical like the housing 18. In the embodiment shown in FIG. 1, the elongate members 14, 16 are attached to each other at or near a proximal end 19 of the housing 18, and are separated from each other at or near a distal end 21 of the housing 18. In this embodiment, the elongate members 14, 16 are cantilevered from a base 23 disposed toward the proximal end 19 of the housing 18.

The identification system 10 also includes an identification feature 20, which is connected to the attachment feature 12 and includes a surface 22 for receiving identifying indicia related to the medical device. This may be, for example, a lot number, a serial number, some other identifying label, or a combination of any of these. The identifying indicia may be laser engraved, etched, or otherwise placed in or on the surface 22 so that it will be available until the associated medical device is used. In at least some embodiments, the identification system 10 can be molded as a single piece from a polymeric material, such as a polymer from the polyaryletherketone (PAEK) family. Polymers from this family may withstand repeated sterilization by autoclaving or other sterilizing processes. The identification system 10 may be made from other materials, for example, a heat-stabilized polypropylene, or any other material effective to provide the properties necessary for its intended uses.

FIG. 2 shows a cross-sectional view of the identification system 10 as viewed through the cut section 2-2 shown in FIG. 1. Shown in FIG. 2 in phantom is a bone screw 24 captured by the elongate members 14, 16 of the attachment feature 12. As shown in FIG. 2, the elongate members 14, 16 together form a generally trapezoidal shape 26 configured to closely resemble the cross section of a head 27 of the screw 24. Also shown in FIG. 2 is a release feature 28 made up of two ramped tabs 30, 32. The tabs 30, 32 are disposed along the insides of their respective elongate members 14, 16. The tabs 30, 32 may extend circumferentially around most or all of the inside portion of their respective elongate members 14, 16, or alternatively may extend for a small portion only along the insides of the elongate members 14, 16.

In practice, the screw 24 will be attached to the identification system 10 prior to sterilization, and then will be placed in a “caddy”, which is brought into the surgical suite to provide ready access to the devices by the surgeon. In order to remove the screw 24 from the identification system 10 and make it ready for use in a patient, a driver may be inserted into a proximal opening 34 at the top of the attachment feature 12. The screw 24 and the identification system 10 may be positioned in a blind hole in the caddy so that as the driver is inserted through the proximal opening 34, it contacts the two ramped tabs 30, 32 and pushes them outward as indicated by the directional arrows 36, 38. To facilitate the outward movement, the tabs 30, 32 have surfaces 33, 35, which are disposed at an oblique angle to an inside surface 37 of the housing 18. Because it is in a blind hole in the caddy, the screw 24 will bottom-out as the elongate members 14, 16 release the screw. This allows the driver to be inserted into the head 27 of the screw 24.

Identification systems, such as the identification system 10, may be made in different sizes to accommodate various medical devices. In particular, a receiving portion, such as the trapezoidal-shaped portion 26, may be sized to receive bone screws of different sizes—for example, bone screws having diameters of 2.5 millimeters (mm), 3 mm, 4 mm, 4.5 mm, etc. Then, to accommodate different sizes of drivers, a bore 39 may have an inside diameter that is dimensioned such that there is a close fit between the walls of the bore 39 and the outside diameter of the driver shaft. This allows the driver shaft to contact and actuate a release feature, such as the tabs 30, 32, merely by inserting it into the bore 39.

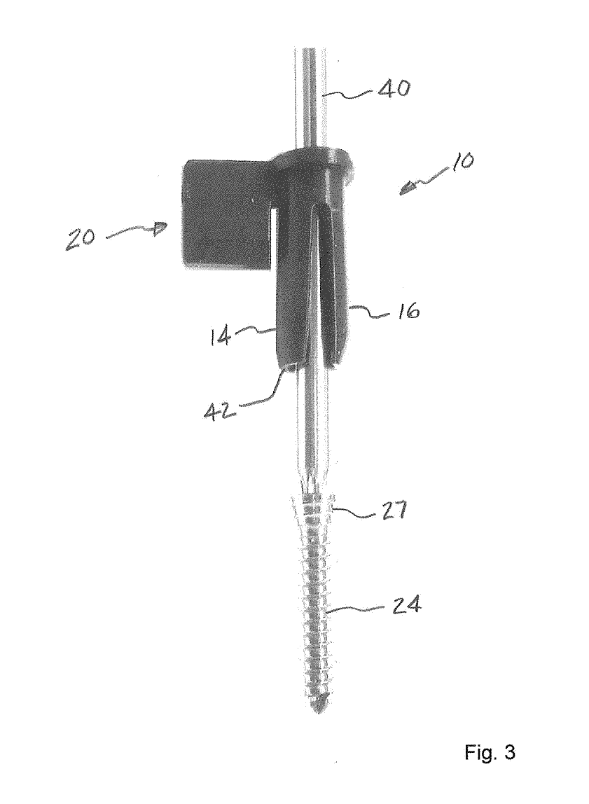

In at least some embodiments, a screw head, such as the screw head 27 may include a hexalobular socket—e.g., a “Torx” head—which facilitates a tight connection between the screw head and the driver. It is therefore possible for the surgeon to remove the screw from the hole in the caddy merely by removing the driver. This is illustrated in FIG. 3, which shows the screw 24 attached tightly to a driver 40. As shown in FIG. 3, the driver 40 has caused the elongate members 14, 16 to separate far enough for the screw head 27 to be released. The depth of the hole in the caddy can be configured to allow a driver, such as the driver 40, to contact and connect with the screw head at some predetermined distance from a distal end 42 of the identification system 10. Once having implanted the screw 24 in a patient, the identification system 10 can be easily removed from the driver 40 and the identifying information provided on the identification feature 20 can be recorded.

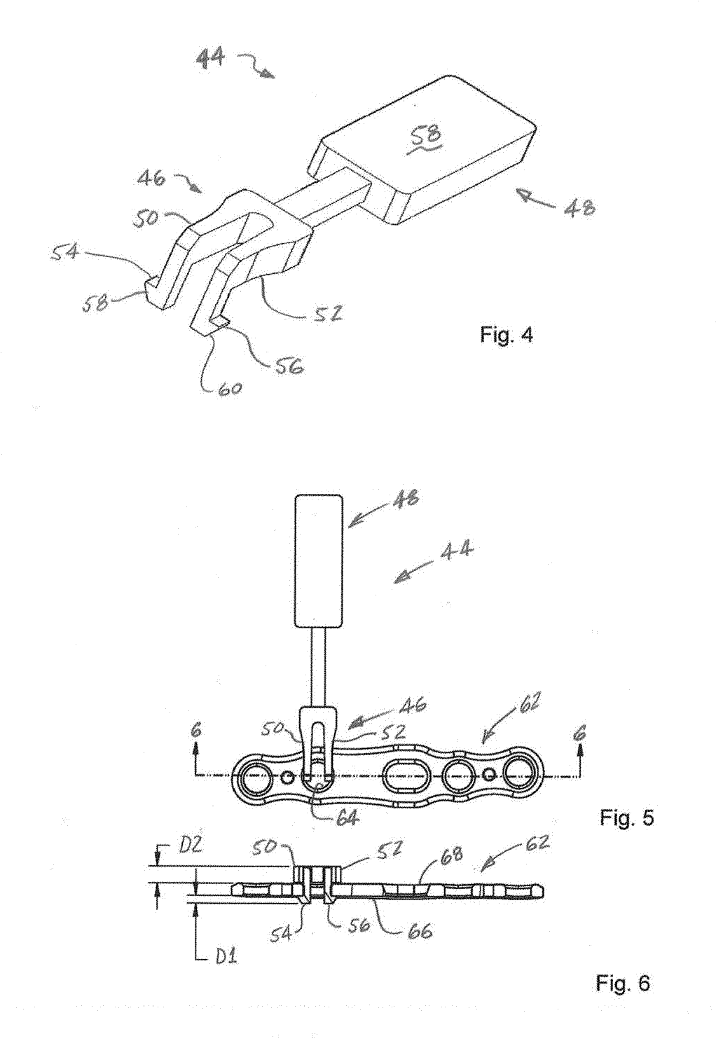

Embodiments of the present invention may also be used with other types of medical devices. FIG. 4 shows a medical identification system 44 in accordance with at least some embodiments described herein. The identification system 44 includes an attachment feature 46 and an identification feature 48. The attachment feature 46 includes a plurality of spaced-apart elongate members 50, 52. Each of the elongate members 50, 52 includes a respective clip 54, 56 transversely extending outwardly therefrom. The clips 54, 56 respectively include ramped portions 58, 60, which, as explained in conjunction with FIG. 5 and FIG. 6, facilitate insertion of the attachment feature 46 into a medical device, such as, for example, a bone plate. Similar to the identification system 10, the identification feature 48 of the identification system 44 includes a surface 58 for receiving identifying information thereon. The identification system 44 may also be made from a PAEK polymer, or any other material effective for its intended uses.

FIG. 5 shows the identification system 44 attached to a bone plate 62. Specifically, the elongate members 50, 52 are inserted into a hole 64 in the bone plate 62. If the hole 64 will be used by the surgeon as part of the fixation, the identification system 44 may be removed before the bone plate 62 is implanted. As described above, the clips 54, 56 include ramped surfaces 58, 60, which allow the identification system 44 to be pressed downward into the hole 64 without the need to separately press the elongate members 50, 52 toward each other. When the ramped surfaces 58, 60 contact the edges of the hole 64, the elongate members 50, 52 will be automatically pushed inward, and will extend outward once the clips 54, 56 are past a distal surface 66 of the bone plate 62—see FIG. 6.

FIG. 6 shows a cross-sectional view of the bone plate 62 and the identification system 44 as viewed through the cut section 6-6 shown in FIG. 5. In the embodiment shown in FIG. 6, the tabs 54, 56 are shown to extend beyond the distal surface 66 of the bone plate 62 by a dimension (D1). In some embodiments, the dimension (D1) may be approximately 0.9 mm, although it could be more or less depending on the size of the bone plate or other medical device to which the identification system 44 is attached. The thickness of the identification system 44 that extends above a proximal surface 68 of the bone plate 62 is shown by dimension (D2). In some embodiments, the dimension (D2) may be approximately 2 mm, although it could be more or less depending on the size of the bone plate or other medical device to which the identification system 44 is attached. Although the medical devices described and illustrated herein consist of a bone screw and a bone plate, identification systems in accordance with embodiments described herein may be configured to attach to other types of medical devices as well.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A medical device identification system, comprising:
an attachment feature including:
a plurality of spaced-apart elongate members configured for removable attachment to a medical device, each of the elongate members forming a portion of a housing configured to receive a tissue fastener, and
a release feature configured to cooperate with a tool for attaching the tissue fastener to tissue such that inserting the tool into the tissue fastener when the tissue fastener is disposed in the housing actuates the release feature to release the tissue fastener from the housing; and
an identification feature connected to the attachment feature and including a surface for receiving identifying indicia related to the medical device.

2. The medical device identification system of claim 1, wherein the attachment feature includes two of the elongate members, and the housing is generally cylindrical.

3. The medical device identification system of claim 1, wherein the release feature includes a plurality of tabs, each of the tabs being attached to a respective one of the elongate members.

4. The medical device identification system of claim 3, wherein each of the tabs includes a surface disposed at an oblique angle to an inside surface of the housing.

5. The medical device identification system of claim 1, wherein at least two of the elongate members are connected to each other toward a proximal end of the housing and are separated from each other toward a distal end of the housing.

6. The medical device identification system of claim 1, wherein the housing includes a base disposed toward a proximal end of the housing, and at least two of the elongate members are cantilevered from the base.
